(19) United States
(12) Patent Application Publication
(10) Pub. No.: US 2025/0356961 A1
(43) Pub. Date: Nov. 20, 2025

(54) OIL SATURATION DELIMITING METHOD WITH JOINTLY PROCESSING OF NUCLEAR MAGNETIC RESONANT RESPONSE (NMR) DATA OF OIL AND ROCK SAMPLES IN IN NATURA CONDITION

(71) Applicant: PETRÓLEO BRASILEIRO S.A. - PETROBRAS, Rio de Janeiro (BR)

(72) Inventor: Moacyr Silva Do Nascimento Neto, Rio de Janiero (BR)

(21) Appl. No.: 19/206,628

(22) Filed: May 13, 2025

(30) Foreign Application Priority Data
May 17, 2024 (BR) BR 1020240098390

(51) Int. Cl. G16C 20/70; G16C 20/20

(52) U.S. Cl. CPC G16C 20/70; G16C 20/20

(57) ABSTRACT

The present invention comprises a method for jointly processing data from the nuclear magnetic resonant (NMR) response of oil and rock samples in in natura condition, and its main product is the delimitation of the oil saturation (Sₒ) present in the rock samples, i.e., the determination of the range of admissible values of the volumetric fraction of the porous volume of the rock occupied by such fluid. The method requires a small volume of oil, originating from or of a composition similar to the oil present in the rock being tested, since it is based on both the measurement of the NMR response of the rock sample and the response of the fluid sample. These two sets of data feed a statistical inference scheme that (1) determines a range of admissible values for the Sₒ of the sample, delimited by the minimum and maximum oil saturation values compatible with the two sets of data presented; (2) produces, for each of these extreme values of admissible Sₒ, a decomposition of the rock relaxation time spectrum into two complementary spectra, the first highlighting the signatures relating to oil and the second comprising the distribution of the rock relaxometric signatures that cannot correspond to such fluid under the prescribed saturation condition.

### FIELD OF THE INVENTION

The present invention is part of the technical field of oil and gas, specifically related to modeling, simulation and evaluation of oil reservoirs, and refers to a method for delimiting oil saturation (Sₒ) with the jointly processing of data from the nuclear magnetic resonant (NMR) response of oil and rock samples in in natura condition.

### BACKGROUND OF THE INVENTION

The identification and quantification of the fluid contents of a geological formation based on data collected in situ is a difficult and complex activity, especially when it comes to classifying the volumes of water and hydrocarbons in an oil reservoir. Typically, decisions in the context of exploration and production of these deposits are supported by measurements made by tools run along the drilled wells (log).

These measurements are commonly restricted to physical amounts that are not of direct interest but are used to infer properties and characteristics of real interest of the geological formation, such as its porosity, hydrocarbon saturation (oil or gas) and its permeability. Log data are often, or need to be, supplemented by another set of measurements of the same physical amounts, acquired, however, under the more rigorous and precise experimental context of laboratories.

To make this possible, physical sampling of rock samples and formation fluids is used at specific intervals in the well. These samples are then conditioned according to the purpose of their analysis or their nature and sent to the competent laboratories.

Among the physical processes tested both in the log and laboratory context, the NMR response of the fluids of a formation stands out as one of the most relevant data for the identification, classification and quantification of its content. An NMR test, in any experimental context, is capable of separating and measuring the magnetization of the atomic nuclei present only in the fluids of a formation when it is subjected to magnetic fields.

In particular, the dynamics of the magnetization of hydrogen nuclei (spin nuclear) present in the water molecules of the formation brine and in the hydrocarbon molecules are observed. The equilibrium nuclear magnetization of each fluid is proportional to its mass and, for this reason, the observation of the total nuclear magnetization provides an indirect way of determining the porosity of the formation (since the fluids present in a geological stratum can only occupy the interstices of its rocks).

On the other hand, separating from this observation the contribution relative to each type of fluid is particularly difficult, because NMR is a technique sensitive to the nuclear spins of all fluids collectively; the acquisition of the NMR response isolated from them cannot be commonly performed on rock samples. This aspect of NMR data acquisition makes its analysis particularly difficult with regard to identifying and quantifying the contribution of each fluid, thus making it difficult to use the NMR response as a procedure for estimating fluid saturations, although, as already stated, the techniques involved are quite sensitive to these amounts.

A widely used strategy for this purpose consists of observing the entire NMR response of the system. Instead of limiting oneself to the magnitude of the NMR signal, from which the equilibrium nuclear magnetization is extracted, the entire evolution of the response is monitored from its activation to its cessation, that is, the entire development of the polarization, or the complete decay of the nuclear magnetization, depending on the experimental protocol.

Nuclear spins exhibit a precession movement around the axis defined by the applied static magnetic field. Under a given static field magnitude, Bₒ, the frequency of this movement is an identifying attribute of the nuclear species, called the Larmor frequency, ω₀. The application of a static field forces the nuclear spins, which are essentially magnetic dipoles, to align with the applied field. This process establishes the nuclear magnetization and has a characteristic time, denoted by T₁, roughly speaking, associated with each molecular species that harbors spins of the probed nuclear species.

Thus, the nuclear magnetic moment of hydrogens residing in water molecules has a polarization time (or longitudinal relaxation) that is different from the nuclear magnetic moment of hydrogens associated with a given chemical component of petroleum, such as paraffins. In this way, each molecular class present in the fluids of a formation can be associated with a nuclear magnetization and distinguished by its characteristic polarization time. This principle is summarized in the model equation:

$$M ( t ) = {\sum }_{\text{?}} {m}_{\text{?}} ( 1 - {\text{?}}^{- \frac{\text{?}}{{T}_{1 , i}}} ) \qquad ( 1 )$$

$$\text{?} \text{indicates text missing or illegible when filed}$$

wherein M(t) represents the total nuclear magnetization observed at a time t and mᵢ and T_{1,i} denote, respectively, the equilibrium nuclear magnetization to the applied field and the longitudinal relaxation time associated with the spins present in the class of molecules indexed by i. The amplitude of the NMR response, that is, the total magnetization of the nuclear spins, is observed after a much longer time (operational five times longer) than the longest relaxation time present in the sample.

This time is denoted here as t→? in agreement with the limit of Equation (1) and the final or equilibrium magnetization of the spins by M_{eq}. FIG. 1 demonstrates the development of an NMR polarization in a rock sample, in which one can clearly identify the stabilization of the acquired signal at a level that determines the M_{eq} in arbitrary units (u.a.) in the test in question.

### The Inversion Problem

Although a classification of the fluids contained in a geological formation can, in theory, be made based on an inversion of the NMR response of the system, in practice, such a procedure encounters some difficulties. The inversion problem consists of determining the unknowns {mᵢ} and {T_{1,i}} from a finite set of observations of the response M(t) at specific instants.

An obvious difficulty is that, even in a perfect inversion, distinct molecules may present similar relaxation times and it is not known, a priori, whether such a coincidence in fact introduces imprecision into the analysis process. Furthermore, it is impossible to distinguish with absolute certainty the molecular species in these situations, since the relaxation times are natural indexes only of the relaxometric classes. Other difficulties present reside (1) in the non-linearity of the relaxation times, Tᵢ, with the system response, (2) in the coupling of these parameters with the magnetizations mᵢ, and (3) in the typical lack of knowledge of the number of relaxometric species present in the system (the number of indexes i).

All these issues, however, are resolved by replacing the discrete model of Equation (1) with the continuous model:

$$M ( t ) = {\int }_{{T}_{1 , min}}^{{T}_{1 , max}} {dT}_{1} ( 1 - {e}^{- \frac{\text{?}}{{T}_{1}}} ) m ( {T}_{1} ) \qquad ( 2 )$$

$$\text{?} \text{indicates text missing or illegible when filed}$$

wherein T_{1,min} and T_{1,max} represent the limits of the band of relaxation times admissible to the system (such limits are typically known or determined by test characteristics). This simple replacement of the model is effective because the relaxation times are no longer unknown, since the entire admissible band of times is covered, and are used as indexes of the object of the inverse problem: the distribution m(T₁).

More technically, neither the acquisition of the NMR response of the system, nor the spectrum of relaxation times associated with it are continuous objects; in any test, the detection of the signal is only performed over a discrete and finite set of observation times, {tᵢ}, and the spectrum is more easily conceived and effectively computed over a partitioning of the admissible band of relaxation times into sub-bands. The model of Equation (2) thus discretized is described by

$${M}_{i} = M ( {t}_{i} ) = {\sum }_{j = 1}^{N} {K}_{ij} {m}_{j} ⇔ {\underbrace{M = Km}}_{Matrix Equation} \qquad ( 3 )$$

$$wherein$$

$${K}_{ij} = \frac{1}{Δ {T}_{i , j}} {\int }_{{T}_{i , j - 1}}^{{T}_{i , j}} {dT}_{1} ( 1 - \text{?} ) \qquad ( 4 )$$

$$denotes the discretized kernel of the inversion and$$

$${m}_{j} = \text{?} {dT}_{1} m ( {T}_{1} ) \qquad ( 5 )$$

$$\text{?} \text{indicates text missing or illegible when filed}$$

wherein (5) represents the nuclear magnetization over the sub-band [T_{i,j-1}, T_{i,j}] of relaxation times. In particular, T_{1,0}=T_{1,min} and T_{1,N}=T_{1,max}.

An inversion of Equation (3), on the other hand, is a difficult task, largely because the kernel of the discretized problem, K, is often a non-invertible matrix, which means that there is more than one object m compatible with the observation M. Therefore, the particular choice of a viable object as the spectrum of the system requires more information about the latter than is contemplated in the experimental observation and depends specifically on the methodology with which this additional information is used to mark such an object as the unique solution of Equation (3) that also satisfies all the a priori knowledge (observation-independent) available about the spectrum.

The non-negativity condition, mⱼ≥0 for all j (represented compactly as m≥0), is an example of this type of complementary information commonly used in the inversion methods pertinent to the problem in question.

In addition to this issue of non-uniqueness, inverse problems such as the one presented also suffer from an issue regarding the non-existence of solutions, often attributed to acquisition noise or other deviations from the real response of the system, such as modeling errors or errors arising from the discretization of the model. It is then established that the acquired data is related to the ideal observation of the system according to:

$$\text{?} = \text{?} + \text{?} \qquad ( 6 )$$

$$\text{?} \text{indicates text missing or illegible when filed}$$

wherein η represents the disturbance of the (ideal) response in the observations considered by the data. Such deviations are strictly unknown; all the information available about them is typically statistical in nature and it is precisely this lack of knowledge about them that makes them one of the most problematic elements of inverse problems.

It is not uncommon for there to be situations wherein the noise level exceeds the intensity of the system response over a range of observations relevant to the problem, which results in obtaining data that is less supported or adequate for the assumed model and consequently in an inconsistent inversion, when M^{˜} is taken indiscriminately as a good approximation for M.

Finally, although the problem was motivated by the observation of the development of nuclear polarization (longitudinal relaxation), the explanation extends to the analysis of several other NMR response tests by redefining the kernel, such as:
transverse relaxation or T₂

$$M ( t ) = {\int }_{{T}_{2 , min}}^{{T}_{2 , max}} {dT}_{2} \text{?} m ( {T}_{2} ) \qquad ( 7 )$$

$$\text{?} \text{indicates text missing or illegible when filed}$$

inversion recovery or IR-T₁

$$M ( t ) = {\int }_{{T}_{1 , min}}^{{T}_{1 , max}} {dT}_{1} ( 1 - 2 \text{?} ) m ( {T}_{1} ) \qquad ( 8 )$$

$$\text{?} \text{indicates text missing or illegible when filed}$$

NMR diffusometry

$$M ( b ) = {\int }_{{D}_{min}}^{{D}_{max}} {dDe}^{- Db} m ( D ) \qquad ( 9 )$$

wherein b denotes the b-value of the acquisition, a continuous experimental control parameter.

### Inversion Methodologies

There are several ways in which the imprecision of measurements can be considered in the inversion method so that the solutions found are consistent and compatible with the degree of fidelity of the observations to the ideal response of the system. Specifically for the class of conditioned linear inverse problems to which the inversion of Equation (3) belongs, the best-established methodology is the regularized minimization of the mean square of the residuals (MSR):

$$Minimize {‖ Km - \text{?} ‖}_{2}^{2} + {α}^{2} {‖ m ‖}_{2}^{2} \qquad ( 10 )$$

$$subject to m \geq 0.$$

$$\text{?} \text{indicates text missing or illegible when filed}$$

wherein ∥·∥₂ denotes the Euclidean norm of the object. The regularization is imposed by the additional term proportional to the quadratic norm of the spectrum and is necessary because the direct minimization of the MSR produces unreasonable solutions, a behavior that, in fact, attests to a lack of conditioning of the inversion program without it. One of the dilemmas introduced by regularization is precisely in the optimal choice of the regularization level (value of α), which ideally is determined by the noise level in the data.

Inference methods comprise another paradigm that enjoys popularity. Entropy Maximization (MaxEnt) is a fundamental axiom of Information Theory, which assumes that the distribution (spectrum) that best meets the set of imposed restrictions (observation, non-negativity, normalization) is obtained without introducing unjustified biases. A classic MaxEnt formulation consists of the program:

$$Maximize - {\sum }_{j} {m}_{j} ln {m}_{j} , \qquad ( 11 )$$

$$subject to m \geq 0$$

$${\sum }_{j} {m}_{j} = {M}_{eq}$$

$$Km = \text{?}$$

$$\text{?} \text{indicates text missing or illegible when filed}$$

which is self-regularizable (does not require searching for the level of regularization). However, the conditioning of the experimental observation to the inversion, when done in terms of equality constraints, as shown, makes the program quite sensitive to the level of noise in the data. Obviously, other constraints, such as the limitation of residuals or the MSR, are perfectly reasonable within the methodology.

### Signature Classification

In addition to the difficulties inherent in the inversion of the NMR response, the classification of the discrete spectrum entries, m, into components specifically associated with one or another fluid is frustrated due to a mechanism of differentiation and shortening of relaxation times, which is quite relevant for water molecules in the regions of the porous volume where the fluid adheres to the surfaces of the mineral framework of the rocks.

The process is known as surface relaxivity, is induced by the presence of paramagnetic impurities housed in the solid interface of the matrix and overcomes the material (or intrinsic) relaxation of a molecular class when, simultaneously, the molecules (1) are polar (i.e., they have an electric dipole), (2) have high mobility within the fluid phase wherein they are inserted (effective molecular transport), and (3) can come into contact with the pore surface and remain confined to relatively narrow regions of the pore volume (such as small pores, surface coating films, menisci or small pockets of fluid adhered to the interface).

The first two conditions are more relevant in the water molecules of the brine of the formation than in the molecules present in oil, whose typical constitution is predominantly marked by nonpolar and low mobility molecules. The third situation is determined by the preferential wettability of the formation, consequently, it can be valid for molecules of all fluids to some degree.

As stated, the three conditions must be valid for a considerable dispersion of relaxation times of the same molecular class to be observed. A simplification, fundamental to the present invention, therefore, is that only water molecules are sensitive to the surface relaxivity mechanism and, therefore, any NMR response related to hydrocarbons retains its intrinsic characteristics. This hypothesis allows the response of petroleum to be distinguished from that of the rest of the formation in situations where a sample of the fluid is accessible for an isolated observation of its signature, as shown in FIG. 2.

In the laboratory context, this is often possible either by extracting a small volume of fluid directly from the rock sample or by using a fluid sample originally collected in situ for chemical analysis or tracing purposes. The NMR response of oil in a geological formation varies little with collection depth, which allows the same fluid sample to be used in the analysis of several rock samples from the same well.

Knowledge of the NMR response of hydrocarbons in the formation allows this information to be highlighted in Equation (1), and the oil saturation level, Sₒ, and, complementarily, the saturation of all other fluids, Sₙₒ, (no denotes non-oil) to be introduced directly into the modeling. In terms of observations, there is

$$M = {M}^{( o )} + {M}^{( no )} \qquad ( 12 )$$

wherein, again, Mₙₒ responds only to molecular classes absent in the oil phase of the formation, that is, they correspond to the non-oil phase (no). Due to the linearity of the response model, Equation (3), it is also possible to transfer this separability to the spectra,

$$m = {m}^{( o )} + {m}^{( no )} . \qquad ( 13 )$$

An entry mⱼ of the spectrum of a sample quantifies the number of nuclear spins that present relaxation times within the range [T_{1,j-1}, T_{1,j}]; in this way, the entire spectrum can be normalized by the number of spins of the sample (equilibrium nuclear magnetization) so that each entry of the normalized spectrum, x, denotes the frequency or relative abundance with which spins relaxing in the corresponding range (relaxometric class) are observed in the sample. That is, it is defined:

$${x}_{j} = \frac{{m}_{j}}{\text{?}} . \qquad ( 14 )$$

$$\text{?} \text{indicates text missing or illegible when filed}$$

From the perspective of Equation (2) and by the definition of the discrete spectrum, Equation (5), the property is directly deduced from the equation above:

$${\sum }_{j = 1}^{N} {x}_{j} = 1 , \qquad ( 15 )$$

wherein, together with the non-negativity of the spectrum (xⱼ≥0 for all j), they constitute the basic set of information, a priori, that is available on the normalized spectrum and that allows it to be taken as the probability distribution of a categorical random variable: the relaxometric class to which any spin in the sample in question may be associated.

It is clear that the normalization of the spectrum can be performed on each of the response phases of the sample, therefore,

$${x}_{j} = \text{?} {x}_{j \vert o} + \text{?} {x}_{j \vert no} . \qquad ( 16 )$$

$$\text{?} \text{indicates text missing or illegible when filed}$$

An important reinterpretation of Equation (13), motivated by the relationship above, is that, in the case of multi-component systems, such as geological formations, xⱼ consists of the relative abundance in the class j of spins on all components of the system, that is,

$${x}_{j} = {\sum }_{α} {X}_{j α} \qquad ( 17 )$$

wherein the index α denotes these components (oil and non-oil, for example). Therefore, for the problem of classifying the signatures present in an NMR response, the specific compositional profile of the sample, whose entries, Xjα, denote the jointly relative abundance of class j (spins that relax with times within the sub-range [Tⱼ₋₁, Tⱼ] for example) and of the α component (oil or non-oil) in the composition, becomes the fundamental object of the analysis, since all the relevant information for the description of the system results from it.

For example, the relative abundance of each component, xα (Sₒ or Sₙo in the particular problem), is defined by:

$${x}_{α} = {\sum }_{j = 1}^{N} {X}_{j α} , \qquad ( 18 )$$

being obviously normalized, since

$${\sum }_{α} {x}_{α} = {\sum }_{α , j} {X}_{j α} = 1. \qquad ( 19 )$$

Furthermore, the relative abundance of a class j specifically in a given component, that is, the normalized spectrum of the phase (xα>0 by hypothesis) is defined by the ratio:

$${x}_{j \vert α} = \frac{{X}_{j α}}{{x}_{α}} . \qquad ( 20 )$$

(j|α reads j given α) for all j with fixed α and, by construction, we have for all α:

$${\sum }_{j} {x}_{j \vert α} = 1 \qquad ( 21 )$$

In view of the above, in order to solve the limitations and technical problems described above, the present invention describes a solution to the problem of classifying the NMR responses of in natura rock samples, which allows it to delimit the oil saturation, Sₒ, in the sample. It only requires the provision of two independent sets of data, M^{˜} and M^{˜}o, respectively related to the NMR responses of the rock and the oil of a formation.

### STATE OF THE ART

The document CN 113125485 B is part of the general state of the art and describes a method and device for non-destructive measurement of rock sample oil saturation in oil and gas exploration and development. According to the invention, the rock sample oil saturation can be accurately obtained, and in the meantime, the rock sample can be maximally protected against damage. The method processes data obtained from the experiment using a series of formulas, but they are different from those used in the present invention.

In its turn, the document U.S. Pat. No. 7,397,240 B2 describes a method for measuring rock wettability by means of low-field nuclear magnetic resonance, which has application in engineering or development of hydrocarbon reservoirs, or in civil engineering. The method essentially determines the surface of water-wet pores and the surface of oil-wet pores when the sample is saturated with water and oil, by measuring the relaxation times (T₁, T₂) of the sample placed in a nuclear magnetic resonance device, previously brought to various states of water or oil saturation, and calculating the wettability index by combining the values of the relaxation times obtained for the said surfaces.

The document U.S. Ser. No. 11/086,044 B2 describes a method and apparatus for analyzing shale oil to continuously characterize the saturation of adsorbed oil and free oil, which involves calculating the saturation of absorbed oil and free oil and analyzing shale oil in the interval of the well to be analyzed. According to the inventors, the solution performs the quantitative and continuous characterization of the adsorbed oil and free oil of shale oil through well logs.

Finally, the document EP 2765409 B1, which is also part of the general state of the art, describes a method for analyzing rock samples by nuclear magnetic resonance with a constant gradient field. The method mainly involves obtaining measurement data by performing nuclear magnetic resonance measurement in a constant gradient field generated by a magnet, converting the nuclear magnetic resonance data, and calculating the fluid property and rock physical property parameters of the rock sample.

Actually, both the present invention and CN 113125485 B and U.S. Ser. No. 11/086,044 B2 are inventions that propose solutions to a very important problem for the oil industry, namely, the estimation of saturation, or volumetric fraction of oil in a geological formation. These three inventions use the same technique at their core, Nuclear Magnetic Resonance (NMR), and all three understand and make use of the understanding that it is necessary to know in advance the NMR response of an oil in order to state how much of this fluid occupies a given formation or rock sample, typically not exclusively filled with it, based on its corresponding NMR response.

On the other hand, this common attribute, by itself, is unable of outlining the procedure or procedural character of any of the three inventions, because simultaneously knowing the NMR responses of the oil and the formation (whether represented by an in situ reading of a profile or represented by data acquired from a plugged rock sample) is nothing more than the minimum requirement necessary for any technique or method that can undoubtedly differentiate the signature of this fluid from the rest of the formation response. The three inventions therefore have equal merit in this proposal, but they are not similar for that reason.

The most notable difference between these three inventions is in the amount of inputs and processes and in the nature of the procedures required to obtain the determinations that each one aims at, given that there is not even a single way to answer the question that all three propose to resolve, and it is precisely at this point that the merit of the present invention is greatest, since it only requires the strict minimum necessary in its approach: an oil sample, a rock sample containing such or similar oil and the NMR response of both.

Such simplicity obviously comes at a price, the present invention does not presume measuring oil saturation, Sₒ, as CN 113125485 B does, nor to differentiate by estimation which fractions of the pore volume are occupied by adsorbed or free oil (non-adsorbed), as U.S. Ser. No. 11/086,044 B2 does. It simply proves capable of delimiting Sₒ within a range of physically possible values consistent with the available information, more specifically, the present invention determines which are the minimum and maximum values of Sₒ compatible with its minimum required information about the system.

It is understandable that one might then ask what the advantage of this is, given that there are procedures for the straightforward determination of Sₒ, as proposed by invention CN 113125485 B or distillation processes, such as Dean-Stark, these even being required in the process flows of inventions U.S. Pat. No. 7,397,240 B2 and U.S. Ser. No. 11/086,044 B2. But the answer is simple: less time and cost. The asset of the present invention does not lie in the product of its invention, which at first glance seems to be a poor substitute for the actual measurement of the parameter, but in the time and cost required to provide its alternative solution, which, like its requirements, are minimal.

The proposed method is the only one among all the inventions considered capable of offering an answer to the problem of determining Sₒ without requiring an entire expensive and time-consuming flow of processes directly acting on the samples, such as drying, draining, cleaning, resaturation, distillation, multiple sampling, whose effectiveness, by the way, is often conditioned by the nature and condition of the samples tested. Instead, the present invention, in a very original way, provides an answer entirely based on a fast and cheap flow of processes directly acting on the data corresponding to the oil and formation samples.

Furthermore, it is important to highlight that the present invention does not require any manipulation of the samples, other than preservation, after their collection, and there is also the possibility of extracting oil by ultracentrifugation of the rock samples, such procedure being strictly optional when there is availability of an oil sample collected directly from a well in the formation, which is quite typical.

Therefore, unlike inventions CN 113125485 B and U.S. Ser. No. 11/086,044 B2, the present invention indicates itself as a fully non-invasive, non-destructive, ready-to-use method, perfect for starting the characterization queue, as it preserves not only the integrity, but also the state of the sample as received, without altering the conformation of the fluids inside it.

A common protocol in oil and service companies is to subject the same sample, collected from a well, to a series of different tests and assays in order to characterize the greatest number of physical, chemical and geological properties of the sample or formation; the priority in this queue is determined by the level of influence that the procedure of one test has on the result of the next, therefore, the less invasive or destructive a test is, the higher its priority, and the faster the information resulting from its analysis can be made available to those interested.

It is also important to highlight that the present invention confers advantages considering the economic and productivity impact associated with the fact that it allows a robust estimate of the volumetric fraction of the porous volume of geological formations occupied by oil. There is currently no non-destructive method in the laboratory context that can achieve this feat.

In addition to the advantages above, social advantages can be highlighted, since the present invention can be equally useful for estimating oil or fat content in other industrial contexts where NMR is used as a sample characterization technique, for example, in determining the oil content in oilseeds, assuming that the NMR response of the chemical components of the extractable oil in fruits or seeds retains its main characteristics after extraction.

Such information should be important for classifying the economic and social potential of certain crops or batches. Furthermore, the decomposition of the component signatures proposed in the methodology may also be useful in areas such as quality control and monitoring food adulteration.

### BRIEF DESCRIPTION OF THE INVENTION

The present invention comprises a method for jointly processing data from the nuclear magnetic resonant (NMR) response of oil and rock samples in in natura condition, and its main product is the delimitation of the oil saturation (Sₒ) present in the rock samples, i.e., the determination of the range of admissible values of the volumetric fraction of the porous volume of the rock occupied by such fluid. The method requires a small volume of oil, originating from or of a composition similar to the oil present in the rock being tested, since it is based on both the measurement of the NMR response of the rock sample and the response of the fluid sample. These two sets of data feed a statistical inference scheme that (1) determines a range of admissible values for the Sₒ of the sample, delimited by the minimum and maximum oil saturation values compatible with the two sets of data shown; (2) produces, for each of these extreme values of admissible Sₒ, a decomposition of the spectrum of relaxation times of the rock into two complementary spectra, the first highlighting the signatures relating to oil and the second comprising the distribution of the relaxometric signatures of the rock that cannot correspond to such fluid under the prescribed condition of saturation.

### BRIEF DESCRIPTION OF THE FIGURES

In order to obtain a complete and total view of the object of this invention, the figures for which references are made below are indicated.

FIG. 1 represents a graph of the typical evolution of the polarization of nuclear spins in a sample. The level of stabilization of the signal amplitude corresponds to the equilibrium magnetization of the system.

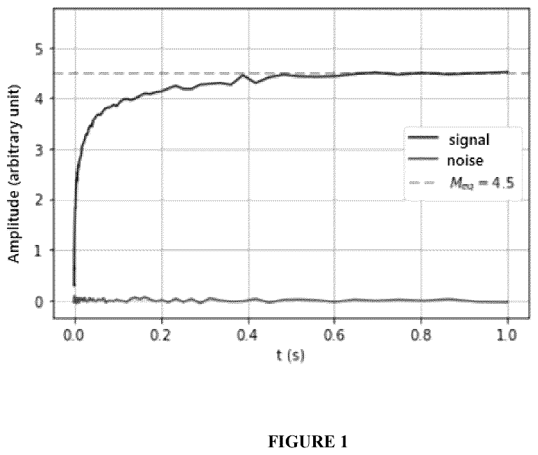

FIG. 2 shows graphs of the T₁ spectra of two rock plugs in contrast to the T₁ spectrum of the same oil sample collected from the same well from which the rock samples originated. The spectra of the rock samples are normalized and that of the oil sample is scaled in order to emphasize the correspondence of the signatures between each pair of samples. The oil sample was collected 50 meters above sample (a) and 70 meters above sample (b), revealing little compositional gradation of the oil in the considered interval of the well.

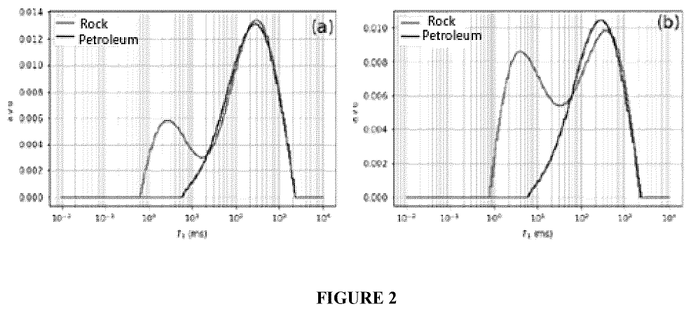

FIG. 3 shows graphs of the Picard Plot for P selection. The data are all related to the same synthetic response; the SNR of each set, however, is different: (a) SNR=1000, (b) SNR=100, (c) SNR=10, (d) SNR=1. It is clear in each graph that there is a stabilization of the singular components of the noise (red points) and a regression of the intersection point of the singular values of the kernel (black points) with the singular components of the signal (blue points) as the noise level increases.

FIG. 4 shows graphs showing the application of the proposed normalization program to the same synthetic T₂ response with different SNRs: (a) SNR=1000, (b) SNR=100, (c) SNR=10, (d) SNR=1. For all decays, M_{eq}=1. The normalization method produces a systematically smaller M_{eq}* for this structure as the data is further deteriorated by noise, while the initial amplitude of the signal fluctuates around the same level.

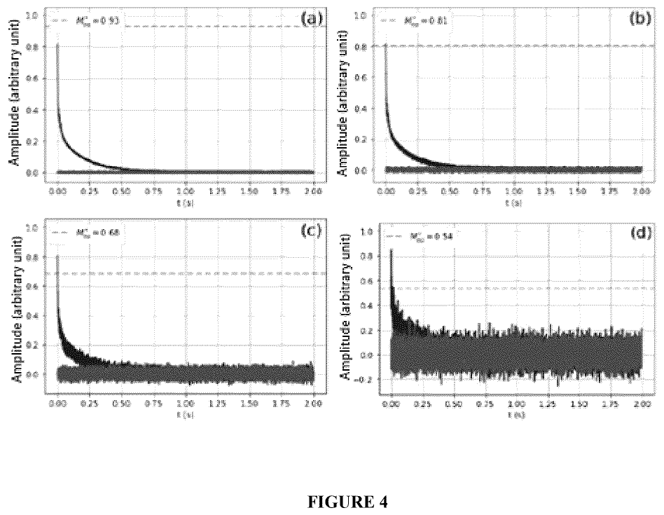

FIG. 5 shows a graph of the entropy 2 S[X] as a function of xₒ, generated from the proposed solution script. For the data set in question, S_{o,min} is determined to be around 40% and S_{o,max}=˜S_{o,max}=76.2%.

FIG. 6 shows graphs of the specific compositional profiles of a raw rock sample, obtained from its longitudinal NMR response: (a) in the condition of S_{o,min}=39.5%; (b) adjustment of the response from the sample spectrum classified in (a), curve in red, with the sample data; (c) in the condition of S_{o,max}=76.2%; (d) adjustment of the response from the sample spectrum classified in (c), curve in red, with the sample data. It is important to highlight that the two spectra generated by each of these compositions, although for the same rock sample, are not in general identical; however, as shown, both fit the data with similar quality (virtually identical R²).

FIG. 7 shows graphs of the specific compositional profiles of a raw rock sample, obtained from its longitudinal NMR response: (a) in the condition of S_{o,min}=20.8%; (b) adjustment of the response from the sample spectrum classified in (a), curve in red, with the sample data; (c) in the condition of S_{o,max}=45.2%; (d) adjustment of the response from the sample spectrum classified in (c), curve in red, with the sample data. It is important to highlight that the two spectra generated by each of these compositions, although for the same rock sample, are not in general identical; however, as shown, both fit the data with similar quality (virtually identical R²).

FIG. 8 shows graphs of the specific compositional profiles of a raw rock sample, obtained from its longitudinal NMR response: (a) in the condition of S_{o,min}=11.1%; (b) adjustment of the response from the sample spectrum classified in (a), curve in red, with the sample data; (c) in the condition of S_{o,max}=20.5%; (d) adjustment of the response from the sample spectrum classified in (c), curve in red, with the sample data. It is important to highlight that the two spectra generated by each of these compositions, although for the same rock sample, are not in general identical; however, as shown, both fit the data with similar quality (virtually identical R²).

FIG. 9 shows graphs of the specific compositional profiles of a raw rock sample, obtained from its transverse NMR response: (a) in the condition of S_{o,min}=23.0%; (b) adjustment of the response from the sample spectrum classified in (a), curve in red, with the sample data; (c) in the condition of S_{o,max}=42.5%; (d) adjustment of the response from the sample spectrum classified in (c), curve in red, with the sample data. It is important to highlight that the two spectra generated by each of these compositions, although for the same rock sample, are not in general identical; however, as shown, both fit the data with similar quality (virtually identical R²).

FIG. 10 shows graphs of the specific-compositional profiles of a natural rock sample, obtained from its transverse NMR response: (a) in the condition of S_{o,min}=39.5%; (b) adjustment of the response from the sample spectrum classified in (a), curve in red, with the sample data; (c) in the condition of S_{o,max}=83.5%; (d) adjustment of the response from the sample spectrum classified in (c), curve in red, with the sample data. It is important to highlight that the two spectra generated by each of these compositions, although for the same rock sample, are not in general identical, however, as shown, both fit the data with similar quality (virtually identical R²).

### DETAILED DESCRIPTION OF THE INVENTION

The present invention provides a solution to the problem of classifying the NMR responses of in natura rock samples, which allows it to delimit the oil saturation, Sₒ, present in the sample. It requires the provision of two independent sets of data, M^{˜} and M^{˜}ₒ, respectively, related to the NMR responses of the rock and the oil of a formation. Since each of the implicit sets of observations is related to a discretized (normalized) spectrum of relaxation times by Equation (3) and assuming that each of these spectra is only one aspect of the specific compositional profile of the sample, the invention directly establishes an inversion routine for X that simultaneously and consistently encompasses all the information made available.

To this end, the method comprises the steps of: (1) discretization of the inversion kernels related to each of the data sets; (2) preconditioning of the linear structure of the problem, Equation (3), via singular value decomposition (SVD); (3) normalization of the data sets; (4) determination of the extreme values of the range of admissible oil saturation values; (5) jointly inversions of the oil and rock data over the entire physically and computationally feasible range of Sₒ values; (6) plot of the specific-compositional profiles of the sample at the minimum and maximum oil saturation levels, respectively S_{o,min} and S_{o,max}, found, highlighting the spectra related to the oil and non-oil phase signatures.

Each of the essential elements and steps of the invention is described below. A data set relating to an NMR response generally consists of a table with three columns of values, namely, the observation parameters (time or b-values), t, and the readings of the signal, M^{˜}, and the observed noise, η^{˜}, (in NMR it is possible to measure the response and a realization of the noise in separate channels).

The entries in η^{˜} do not generally correspond to the deviations from the ideal observation, present in M^{˜}, but they are, admittedly, generated by a random process identical to that which generates them. Thus, η^{˜}, the noise observed directly, is used to infer statistical properties of the noise contaminating the data, such as the mean and standard deviation of the fluctuations and confidence intervals for the residuals.

### Discretization of the Inversion Kernel

K(t, T) is the generic kernel of the continuous model (Equations (7, 8, 9) provide particular cases for this generic kernel) and T is the response classification parameter (T₂, T₁ or D). According to the continuous model, there is:

$${\tilde{M}}_{i} = {\int }_{{T}_{min}}^{{T}_{max}} dTK ( {t}_{i} , T ) m ( T ) + {η}_{i} . \qquad ( 22 )$$

The full discretization of the problem is performed using the Galerkin method wherein the distribution m(T) is projected onto a family of top-hat functions,

$${h}_{j} ( T ) = \{ \begin{matrix}1 & if {T}_{j - 1} < T < {T}_{j} \\ 0 & otherwise\end{matrix} , \qquad ( 23 )$$

wherein the support of each function, that is, the zone of its domain wherein the function is non-zero, is defined over a specific sub-band of the admissible band of the parameter T. In the case of relaxation times, Tₘᵢₙ=10⁻² ms and Tₘₐₓ=10⁴ ms are usually taken and the band partitioning is done by logarithmically spacing a fixed number of sub-bands (typically N=64, 128 or 256). Thus, the continuous distribution is imposed with the form:

$$m ( T ) = {\sum }_{j = 1}^{N} {m}_{j} \frac{{h}_{j} ( T )}{Δ {T}_{j}} + Δ m ( T ) , \qquad ( 24 )$$

with Δm(T) denoting the representation error of the distribution m(T) over the family of top-hat functions considered; Equation (24) is replaced in Equation (22) and the representation error is ignored to obtain Equation (3), that is, the definition of the discretized kernel; each of the Ki7 entries is explicitly calculated for the full discretization of the model. The main advantage of this discretization scheme is that the variables of the inversion problem are the values of the discrete spectrum, Equation (5).

### Preconditioning of the Linear Structure

The discretized kernel is generally a rectangular matrix. The number of observations, equivalent to the number of rows of K, is typically high, which generally leads to redundancy of information about the NMR response in the acquired data. Preconditioning therefore has the function of reducing the size of K, roughly speaking, through a selection (or adoption) of a subset of informationally less dependent data, which makes the final linear structure of the problem more compact, implying gains in computational time and feasibility.

One of the most effective preconditioning techniques is data compression via SVD (singular value decomposition) of the discretized kernel. K is decomposed into its singular value structure, that is:

$$K = U \sum {V}^{†} \qquad ( 25 )$$

where Σ is a diagonal matrix of the same size as K, whose non-zero elements denote the singular values of the kernel and U and V are unitary matrices whose columns denote respectively the singular vectors to the left and right of K; the vectors of singular components of the data and noise are defined, respectively, by the projections:

$${U}^{†} \tilde{M} {eU}^{†} \tilde{η} ;$$

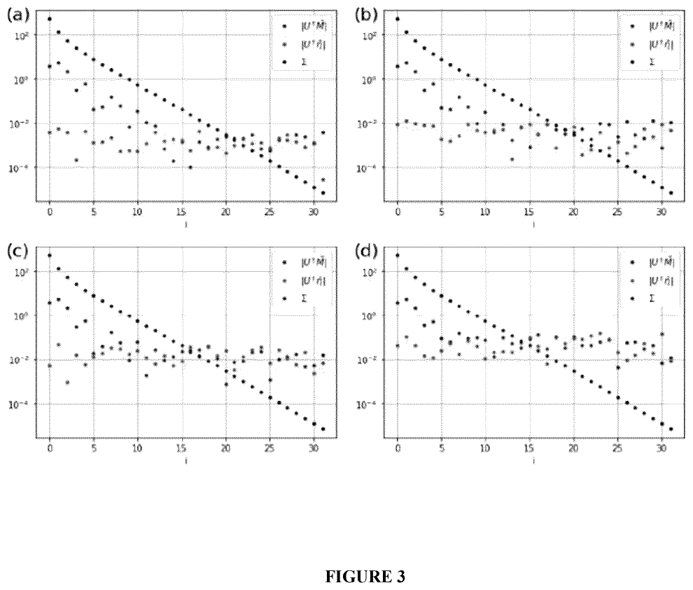

Only a number of singular components of the data are retained equal to the number of singular values of the kernel greater than the level of the singular components of the noise (this requirement is known as the Picard criterion and can be graphically represented, as shown in FIG. 3), such number is designated by P; then it is defined:

$${K}^{( P )} = {\sum }^{( P )} {V}^{†} \qquad ( 26 )$$

$${\tilde{M}}^{( P )} = {{U}^{†}}^{( P )} \tilde{M} \qquad ( 27 )$$

where the superscript (P) indicates truncation of the matrices of the singular structure up to line P; K^{(P)} and M^{˜(P)} are taken by the new linear (compressed) structure of the problem. Note that K^{(P)} thus has size P x N.

In practice, the method indicated here is stable when retaining a greater number of singular components of the data than that determined by the Picard criterion, as long as the increase is not too large. Therefore, a fixed number of singular components is usually adopted regardless of the signal-to-noise ratio (SNR) of the data, P=32, for example, usually serves for data with SNR=1 up to SNR=1000.

### Normalization

The normalization problem consists of estimating M_{eq} from the set of observations. The value of the equilibrium magnetization of the system or, more specifically, the signal volume is an important parameter to be able to introduce the normalization condition, essential in statistical inference approaches, to the inversion method.

In tests where the acquisition noise is acceptable and the signal sampling rate is sufficiently high, M_{eq} is typically estimated by the arithmetic mean of the first points with the highest positive amplitude of the response. In the present invention, a distinct method is proposed that uses all the information in the linear structure of the problem and by which it is guaranteed that M*_{eq}, the estimate, is not necessarily greater than M_{eq}, the real value of the equilibrium magnetization.

The condition M*_{eq}≤M_{eq} ensures that the structure of the inversion problem, normalized by the estimate, has a set of feasible solutions that necessarily contains the set of feasible solutions that would be defined by the correct normalization of the data. Therefore, let m* be the solution of the linear programming (LP) below:

$$Minimize {\sum }_{j = 1}^{N} {m}_{j} \qquad ( 28 )$$

$$subject to {‖ {K}^{( P )} m - {\tilde{M}}^{( P )} ‖}_{∞} \leq {δ}_{η}$$

$$m \geq 0$$

where ∥y∥∞=maxᵢ␣yᵢ| denotes the Chebyshev norm and δη represents a confidence limit for the fluctuation of the residuals: δη=3ση, where ση indicates the standard deviation of the observed noise, in cases where this is normal, establishing the known confidence level greater than 99%. It is defined:

$${M}_{eq}^{*} = {\sum }_{j = 1}^{N} {m}_{j}^{*} \qquad ( 29 )$$

(necessarily there is M*_{eq}≤M_{eq}) and

$${\overline{M}}^{( P )} = \frac{{\tilde{M}}^{( P )}}{{M}_{eq}^{*}} e {\overline{δ}}_{η} = \frac{{δ}_{η}}{{M}_{eq}^{*}} , \qquad ( 30 )$$

Respectively, as the data and the limit of the residuals referring to the normalized spectrum.

### Delimitation of the Limiting Values of Sₒ

The maximization of the quadratic entropy of the specific-compositional profile of the sample is taken as an axiom of inference and, therefore, a principle of inversion. The quadratic entropy of a non-negative and normalized distribution is defined by the expression:

$${}^{2} S [ X ] = 1 - {‖ X ‖}_{2}^{2} . \qquad ( 31 )$$

For the case of interest wherein X represents the specific-compositional profile of a two-phase system, there is:

$${}^{2} S [ X ] = {}^{2} S [ \{ {x}_{o} , {x}_{no} \} ] + {}^{2} S [ {x}_{\vert o} ] {x}_{o}^{2} + {}^{2} S [ {x}_{\vert no} ] {x}_{no}^{2} . \qquad ( 32 )$$

The following inversion program is then used to determine the minimum and maximum values of Sₒ compatible with the data sets of the rock sample and the oil sample:

$$Optimize {}^{2} S [ x ] + {}^{2} S [ {x}_{\vert o} ] {x}_{o}^{2} + {}^{2} S [ {x}_{\vert no} ] {x}_{no}^{2} \qquad ( 33 )$$

$$subject to {x}_{o} \geq 0$$

$${x}_{no} \geq 0$$

$${x}_{o} + {x}_{no} = 1$$

$${x}_{\vert no} = arg [ \begin{matrix}Maximize {}^{2} S [ y ] \\ subject to y \geq 0 \\ \text{?} = 1 \\ ‖ {x}_{no} {K}^{( P )} y - ( \text{?} - {x}_{o} {K}^{( P )} \text{?} ) \text{?} \leq \text{?}\end{matrix} ]$$

$${x}_{\vert o} = arg [ \begin{matrix}Maximize {}^{2} S [ y ] \\ subject to y \geq 0 \\ \text{?} = 1 \\ ‖ \text{?} y - \text{?} \leq \text{?}\end{matrix} ]$$

$$\text{?} \text{indicates text missing or illegible when filed}$$

It is necessary that the two data sets, the rock and the oil, have been preconditioned, but not necessarily to the same level, that is, the two data sets may have different P even if their temporal samplings are identical; it is necessary that the discrete spectra of each phase have the same size for X to be a matrix. This is ensured by using the same family of top-hat functions when applying the Galerkin method for kernel discretization; both data sets also need to be normalized. Finally, the subscript (P) is kept to indicate the structure of the rock sample and (o) is used to denote the structure of the oil sample.

The Optimize directive in the above program should be interpreted as Maximize to produce the estimate of minimum Sₒ, or S_{o,min}, and as Minimize to produce the estimate of maximum Sₒ, or S_{o,max}. In both computations, Sₒ≡x*ₒ, wherein x*ₒ is taken from the solution of each program. Therefore, it is necessary to solve the program in both optimization directions to produce the bounds of the interval of Sₒ compatible with all the available information about the sample.

While the search for S_{o,min} is straightforward, the search for S_{o,max} requires the additional imposition of the consistency condition S_{o,max}≥S_{o,min}. This results from the fact that the first optimization is satisfied by the global maximum of 2 S[X] over the set of feasible objects, while its minimization can lead the solution to tend towards either of the extremes xₒ=0 or xₒ=1.

### Jointly Inversion

Program (33) consists of a two-level optimization problem: an optimization conditioned on another optimization. Such problems are notoriously difficult to solve.

The following script is then proposed for solving the program:
solve the problem of determining the spectrum of the oil, x|o, independently;
solve the problem of determining the spectrum of the sample, x, which corresponds to the case x|no with xo=0;
define

$${\tilde{S}}_{o , max} = \frac{{x}^{†} {x}_{\vert o}}{{x}_{\vert o}^{†} {x}_{\vert o}}$$

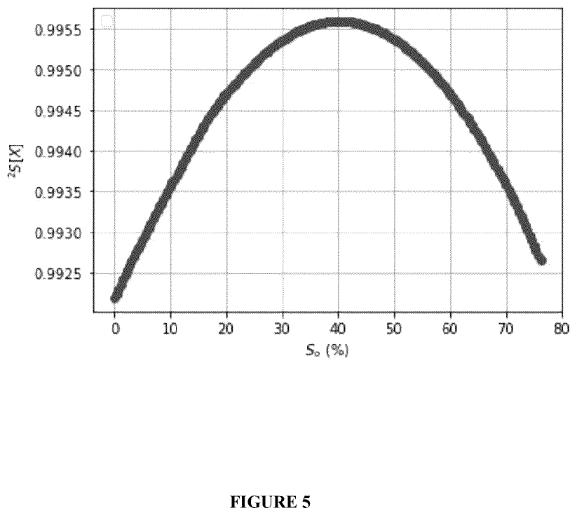

which corresponds to the projection of the spectrum of the sample onto that of the oil;
a list of values for xₒ is fixed covering the entire range of physically acceptable saturations, xₒε[0, S^{˜}_{o,max];}
the program associated with the determination of x|no is solved for each of the values of xₒ (and therefore xno) from the list above, this produces a sequence of spectra x|no or profiles X and values of 2 S[X], as shown in FIG. 5;
S_{o,min} is identified with the value of xₒ from the list for which 2 S[X] is maximum and S_{o,max} with the value of xₒ≥S_{o,min} from the list for which 2 S [X] presents its lowest value.

The above script ends up producing all the specific-compositional profiles of the sample compatible with the information available between S_{o,min} and S_{o,max}. The introduction of an upper bound for xₒ, such as the adopted S^{˜}_{o,max}, is necessary because the minimization program of 2 S [X] may be led to seek values of xₒ outside its feasibility domain, since there is no a priori guarantee that it is possible to find an x|no for any prescribed value of xₒ.

On the other hand, it is always possible to obtain viable x|no for low values of xₒ since such a procedure corresponds to simply associating most of the information contained in the sample data with signatures of the non-oil phase.

In this sense, S_{o,min}, as defined, represents the value of xₒ under which the contributions of the sample signatures attributable to the oil phase are partitioned more equally among the components of the classification. Crossing this threshold, therefore, means weighing this association of signatures of the sample response even more towards the oil phase, until it is possible to identify them in the response of the resulting non-oil phase.

Since entropy maximization consists only of a criterion for selecting the specific-compositional profile that satisfies the imposed conditions, maximizing the number of parametric categories (of the specific-compositional profile), intuitively, it is expected that at S_{o,min} approximately half of the contributions attributable to the oil phase are still associated with the non-oil phase. This notion explains the tendency to obtain values for S_{o,max}≈2S_{o,min} using the method.

### Plot of Specific-Compositional Profiles

X is chosen to be represented graphically as the simultaneous plot of the distributions {Xj,o} and {Xj,no}. Note that each of them consists of the normalized spectrum of each phase, modulated by its respective saturation, that is, Sₒxₒ and Sₙₒxₙₒ respectively. To illustrate the natural tendency of the results, it is only necessary to plot the profiles associated with the S_{o,min} and S_{o,max} found.

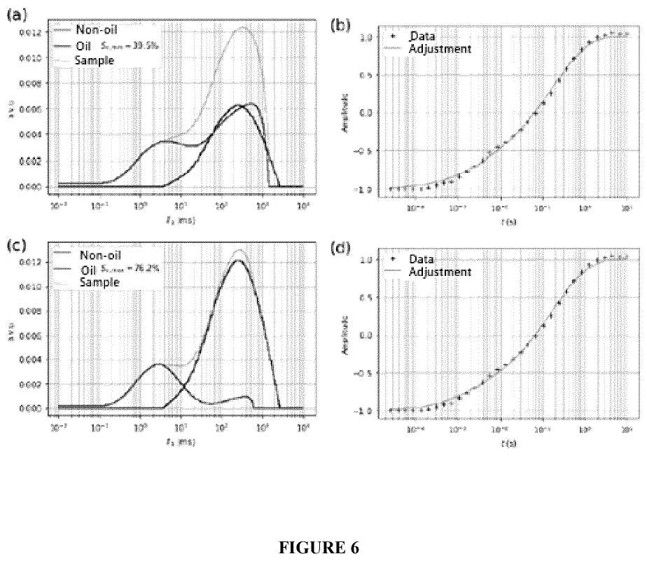

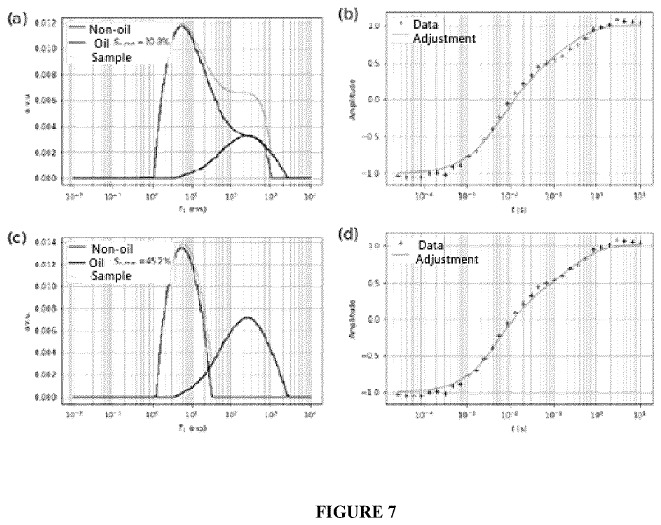

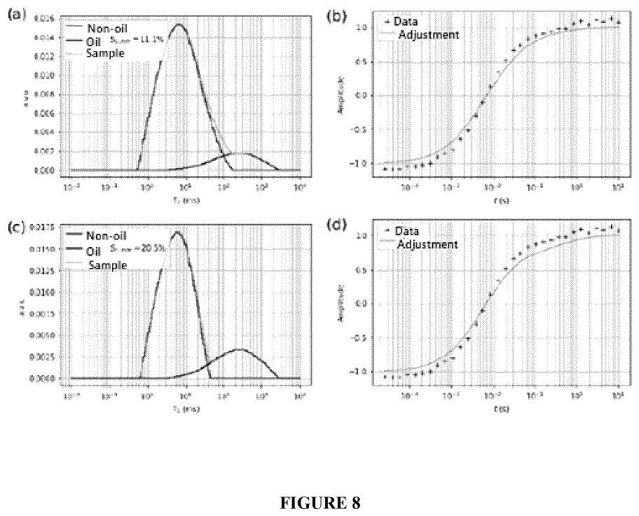

The first three plots represent the profiles of three samples from the same well, generated from the longitudinal NMR response, Inversion Recovery test. The fluid data used in all three classifications is the same, acquired from a single oil sample from the well, collected for chemical analysis and stored for tracking. FIG. 6, FIG. 7, FIG. 8 demonstrate the performance of the method in situations of high, medium and low oil saturation in rock samples, respectively.

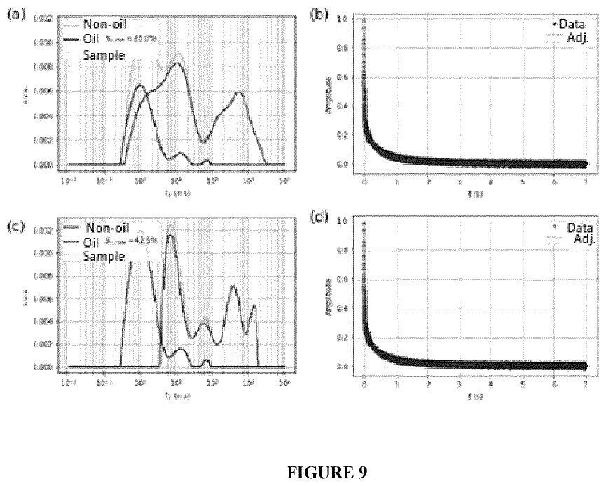

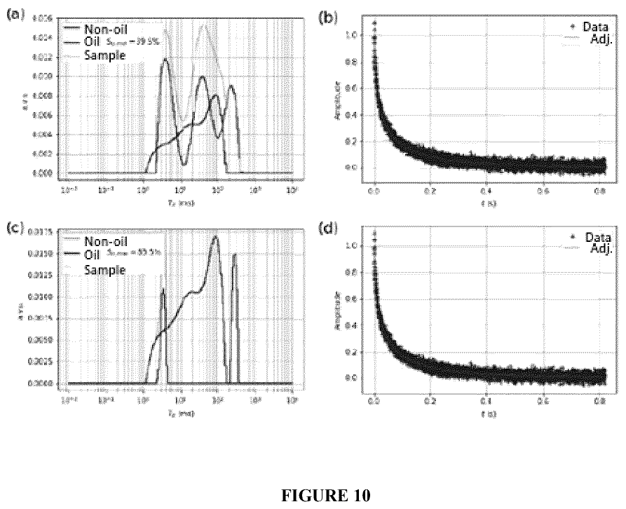

The last two graphs, FIG. 9 and FIG. 10, illustrate the flexibility of the method in using other types of NMR responses, such as those from CPMG tests, where the transverse magnetization of the samples is measured. Alongside the graphical representation of each profile, the quality of the adjustment of the distributions obtained against the acquired data is graphically demonstrated. The R₂ of all adjustments is greater than 98%.

Therefore, when analyzing the figures mentioned above, it is noted that this decomposition of the spectra does not exist in the state of the art. The typical result of rock analyses are the spectra in red (Sample caption in each of the figures). It is usually up to an interpreter to identify in this single curve what corresponds to each fluid phase. The method proposes an automatic decomposition of the sample spectrum (red) into the oil spectrum (black) and the spectrum of non-oil signatures (blue).

When added together, these last two represent the first. However, this decomposition depends on the saturation level of the conditioned oil, so there is a decomposition for each Sₒ value within the range [S_{o,min}, S_{o,max}].

It is chosen to graphically represent only the decomposition in the Sₒ limits. Another thing that the method demonstrates, and for this reason it is important to represent the adjustments of the data next to each decomposition, since there is not only one spectrum that adjusts the observed data. The decompositions generate different spectra for the same sample, when comparing the spectra in red in the S_{o,min} situation.

Those skilled in the art will value the knowledge being shown and will be able to reproduce the invention in the indicated embodiments and in other variants, covered by the scope of the attached claims.

1. A method, for oil saturation delimiting via jointly processing of nuclear magnetic resonant (NMR) response data from oil and rock samples in in natura condition, comprising the steps of:
(1) discretization of inversion kernels related to data sets;
(2) preconditioning of a linear structure of a problem, via singular value decomposition (SVD);
(3) normalization of the data sets;
(4) determination of extreme values of a range of admissible oil saturation values;
(5) jointly inversions of oil and rock data over an entire physically and computationally feasible range of Sₒ values; and
(6) plotting of specific-compositional profiles of a sample at a minimum and maximum oil saturation levels, respectively S_{o,min} and S_{o,max}, found, wherein plotting of the profiles highlights spectra related to signatures of the oil and non-oil phase.

2. The method, according to claim 1, wherein jointly data processing classifies fluid signatures in porous media samples in in natura condition, preferably with an emphasis on identifying and estimating preponderance of oil signatures.

3. The method, according to claim 1, wherein the normalization of the data sets is intended to adapt the linear structure of the problem to a statistical inference paradigm.

4. The method, according to claim 1, wherein in the normalization step it is ensured that M*_{eq} is necessarily not greater than M_{eq}.

5. The method, according to claim 1, wherein searching for S_{o,max} requires additional imposition of consistency condition S_{o,max}≥S_{o,min}.
